(19) United States
(12) Patent Application Publication
(10) Pub. No.: US 2022/0395815 A1
(43) Pub. Date: Dec. 15, 2022

(54) METHOD FOR PREPARATION OF HETEROGENEOUS CATALYSTS

(71) Applicant: Dow Global Technologies LLC, Midland, MI (US)

(72) Inventors: Victor J. Sussman, Midland, MI (US); Wen-Sheng Lee, Midland, MI (US); Jeffrey A. Herron, Midland, MI (US); D. Wayne Blaylock, Fort Bend, TX (US); Daniel J. Arriola, Midland, MI (US); Andrew T. Heitsch, Angleton, TX (US); Alexey Kirilin, Terneuzen (NL); Heidi Clements, Bay City, MI (US); Abrin L. Schmucker, Midland, MI (US); Daniel A. Hickman, Midland, MI (US)

(21) Appl. No.: 17/889,708

(22) Filed: Aug. 17, 2022

Related U.S. Application Data
(63) Continuation of application No. 16/634,413, filed on Jan. 27, 2020.
(60) Provisional application No. 62/538,229, filed on Jul. 28, 2017.

(51) Int. Cl. B01J 23/52; B01J 21/04; B01J 35/00; B01J 35/02; B01J 37/02; B01J 37/04; B01J 37/08

(52) U.S. Cl. CPC B01J 23/52; B01J 21/04; B01J 35/008; B01J 35/023; B01J 35/026; B01J 37/0219; B01J 37/0221; B01J 37/0236; B01J 37/04; B01J 37/086; B01J 37/088

(57) ABSTRACT

A method for preparing a heterogeneous catalyst. The method comprises steps of: (a) combining (i) a support, (ii) an aqueous solution of a noble metal compound and (iii) a C₂-C₁₈ thiol comprising at least one hydroxyl or carboxylic acid substituent; to form a wet particle and (b) removing water from the wet particle by drying followed by calcination to produce the catalyst.

### BACKGROUND OF THE INVENTION

The invention relates to a method for preparing heterogeneous catalysts. The catalysts are especially useful in a process for preparing methyl methacrylate from methacrolein and methanol.

Heterogeneous catalysts made by deposition of metals in the presence of thio acids are known, see, e.g., U.S. Pat. No. 3,972,829. However, there is a need for catalysts which provide improved yield and/or selectivity.

### SUMMARY OF THE INVENTION

The present invention is directed to a method for preparing a heterogeneous catalyst; said method comprising steps of: (a) combining (i) a support, (ii) an aqueous solution of a noble metal compound and (iii) a C₂-C₁₈ thiol comprising at least one hydroxyl or carboxylic acid substituent; to form a wet particle and (b) removing water from the wet particle by drying followed by calcination to produce the catalyst.

### DETAILED DESCRIPTION OF THE INVENTION

All percentage compositions are weight percentages (wt %), and all temperatures are in ° C., unless otherwise indicated. A noble metal is any of gold, platinum, iridium, osmium, silver, palladium, rhodium and ruthenium. More than one noble metal may be present in the catalyst, in which case the limits apply to the total of all noble metals. The “catalyst center” is the centroid of the catalyst particle, i.e., the mean position of all points in all coordinate directions. A diameter is any linear dimension passing through the catalyst center and the average diameter is the arithmetic mean of all possible diameters. The aspect ratio is the ratio of the longest to the shortest diameters.

Preferably, the support is a particle of a refractory oxide; preferably γ-, δ-, or θ-alumina, silica, magnesia, titania, zirconia, hafnia, vanadia, niobium oxide, tantalum oxide, ceria, yttria, lanthanum oxide or a combination thereof; preferably γ-, δ-, or θ-alumina. Preferably, in portions of the catalyst comprising noble metal, the support has a surface area greater than 10 m²/g, preferably greater than 30 m²/g, preferably greater than 50 m²/g, preferably greater than 100 m²/g, preferably greater than 120 m²/g. In portions of the catalyst which comprise little or no noble metal, the support may have a surface area less than 50 m²/g, preferably less than 20 m²/g.

Preferably, the aspect ratio of the catalyst particle is no more than 10:1, preferably no more than 5:1, preferably no more than 3:1, preferably no more than 2:1, preferably no more than 1.5:1, preferably no more than 1.1:1. Preferred shapes for the particle include spheres, cylinders, rectangular solids, rings, multi-lobed shapes (e.g., cloverleaf cross section), shapes having multiple holes and “wagon wheels,” preferably spheres. Irregular shapes may also be used.

Preferably, at least 90 wt % of the noble metal(s) is in the outer 40% of catalyst volume, preferably the outer 35%, preferably in the outer 30%, preferably in the outer 25%. Preferably, the outer volume of any particle shape is calculated for a volume having a constant distance from its inner surface to its outer surface (the surface of the particle), measured along a line perpendicular to the outer surface. For example, for a spherical particle the outer x % of volume is a spherical shell whose outer surface is the surface of the particle and whose volume is x % of the volume of the entire sphere. Preferably, at least 95 wt % of the noble metal is in the outer volume of the catalyst, preferably at least 97 wt %, preferably at least 99 wt %. Preferably, at least 90 wt % (preferably at least 95 wt %, preferably at least 97 wt %, preferably at least 99 wt %) of the noble metal(s) is within a distance from the surface that is no more than 15% of the catalyst diameter, preferably no more than 10%, preferably no more than 8%, preferably no more than 6%. Distance from the surface is measured along a line which is perpendicular to the surface.

Preferably, the noble metal is gold or palladium, preferably gold.

Preferably, the average diameter of the catalyst particle is at least 60 microns, preferably at least 80 microns, preferably at least 100 microns, preferably at least 200 microns, preferably at least 300 microns, preferably at least 400 microns, preferably at least 500 microns, preferably at least 600 microns, preferably at least 700 microns, preferably at least 800 microns; preferably no more than 30 mm, preferably no more than 20 mm, preferably no more than 10 mm, preferably no more than 5 mm, preferably no more than 4 mm, preferably no more than 3 mm The average diameter of the support and the average diameter of the final catalyst particle are not significantly different.

Preferably, the C₂-C₁₈ thiol comprising at least one hydroxyl or carboxylic acid substituent has from 2 to 12 carbon atoms, preferably 2 to 8, preferably 3 to 6. Preferably, the thiol compound comprises no more than 4 total hydroxyl and carboxylic acid groups, preferably no more than 3, preferably no more than 2. Preferably, the thiol compound has no more than 2 thiol groups, preferably no more than one. If the thiol compound comprises carboxylic acid substituents, they may be present in the acid form, conjugate base form or a mixture thereof. The thiol component also may be present either in its thiol (acid) form or its conjugate base (thiolate) form. Especially preferred thiol compounds include thiomalic acid, 3-mercaptopropionic acid, thioglycolic acid, 2-mercaptoethanol and 1-thioglycerol, including their conjugate bases.

Preferably, the catalyst is produced by precipitating the noble metal from an aqueous solution of noble metal salt in the presence of the support. In one preferred embodiment, the catalyst is produced by an incipient wetness technique in which an aqueous solution of a suitable noble metal precursor salt is added to a porous inorganic oxide such that the pores are filled with the solution and the water is then removed by drying. Preferred noble metal salts include tetrachloroauric acid, sodium aurothiosulfate, sodium aurothiomalate, gold hydroxide, palladium nitrate, palladium chloride and palladium acetate. Preferably, the wet particle is dried at a temperature from 20-150° C. at atmospheric pressure or under vacuum, preferably for at least one hour. The resulting material (dried particle) is then converted into a finished catalyst by calcination, reduction, or other treatments known to those skilled in the art to decompose the noble metal salts into metals or metal oxides. Preferably, calcination is performed at a temperature from 200-700° C., preferably at least 250° C., preferably at least 280° C.; preferably no more than 600° C., preferably no more than 550° C., preferably no more than 500° C. Preferably, the time for calcination is from 1-24 hours.

In another preferred embodiment, the catalyst is produced by deposition precipitation in which a porous inorganic oxide is immersed in an aqueous solution containing a suitable noble metal precursor salt and that salt is then made to interact with the surface of the inorganic oxide by adjusting the pH of the solution. The resulting treated solid is then recovered (e.g. by filtration) and then converted into a finished catalyst by calcination, reduction, or other treatments known to those skilled in the art to decompose the noble metal salts into metals or metal oxides.

Amounts of noble metal salt and water are determined by the amount of support and the desired level of noble metal in the catalyst and may be calculated easily by those skilled in the art. Preferably, the amount of noble metal as a percentage of the noble metal and the support is from 0.2 to 5 wt %, preferably at least 0.5 wt %, preferably at least 0.8 wt %, preferably at least 1 wt %, preferably 1.2 wt %; preferably no more than 4 wt %, preferably no more than 3 wt %, preferably no more than 2.5 wt %. Preferably, the ratio of C₂-C₁₈ thiol comprising at least one hydroxyl or carboxylic acid group to noble metal is from 50:1 to 10:1, more preferably from 5:1 to 2:1.

The catalyst of this invention is useful in a process for producing methyl methacrylate (MMA) which comprises treating methacrolein with methanol in an oxidative esterification reactor (OER) containing a catalyst bed. The catalyst bed comprises the catalyst particles and is situated within the OER that liquid flow may occur through the catalyst bed. The catalyst particles in the catalyst bed typically are held in place by solid walls and by screens. In some configurations, the screens are on opposite ends of the catalyst bed and the solid walls are on the side(s), although in some configurations the catalyst bed may be enclosed entirely by screens. Preferred shapes for the catalyst bed include a cylinder, a rectangular solid and a cylindrical shell; preferably a cylinder. The OER further comprises a liquid phase comprising methacrolein, methanol and MMA and a gaseous phase comprising oxygen. The liquid phase may further comprise byproducts, e.g., methacrolein dimethyl acetal (MDA) and methyl isobutyrate (MIB). Preferably, the liquid phase is at a temperature from 40 to 120° C.; preferably at least 50° C., preferably at least 60° C.; preferably no more than 110° C., preferably no more than 100° C. Preferably, the catalyst bed is at a pressure from 0 to 2000 psig (101 kPa to 14 MPa); preferably no more than 2000 kPa, preferably no more than 1500 kPa. Preferably, pH in the catalyst bed is from 4 to 10; preferably at least 4.5, preferably at least 5; preferably no greater than 9, preferably no greater than 8, preferably no greater than 7.5, preferably no greater than 7, preferably no greater than 6.5. Preferably, the catalyst bed is in a tubular continuous reactor or a continuous stirred tank reactor, preferably a tubular continuous reactor.

### EXAMPLES

### A. Sodium Aurothiosulfate Incipient Wetness Catalyst—Powder Version

### A.1—Raw Materials

| | | | | Purity | | |
|---|---|---|---|---|---|---|
| Material Name | Formula (MW) | CAS No. | Amt. | Spec. | Supplier | Description |
| sodium | [Na]₃[Au(S₂O₃)₂]•xH₂O | 15283-45-1 | 3.73 g | 99.9% | Alfa Aesar | white |
| aurothiosulfate | (490.19 anhy.) | | | (metals | Cat No. | powder |
| hydrate | | | | basis) | 39741 | |
| Puralox 5/90 | Al₂O₃ | 1344-28-1 | 100 g | As | Sasol | white |
| Alumina | (101.96) | | | supplied | | powder |
| Distilled Water | H₂O | 7732-18-5 | 86 g | deionized | | — |
| | (18.02) | | | | | |

### A.2—Catalyst Preparation—Target Loading of 1.5 wt % Au

1. Weigh out 100 g of Puralox 5/90 alumina.
2. Dissolve 3.73 g of sodium aurothiosulfate hydrate in 86 g of distilled deionized water to form a clear, colorless solution.
3. Impregnate the Puralox 5/90 alumina by incipient wetness using the solution prepared in #2.
4. Place the impregnated material into a vacuum oven and dry at 80° C. for one hour.
5. Calcine dried catalyst material using a ramp rate of 5°/min to 400° C. and hold at 400° C. for 4 h.

### C. Sodium Aurothiomalate Incipient Wetness Catalyst—Powder Version

### C.1—Raw Materials

| | | | | Purity | | |
|---|---|---|---|---|---|---|
| Material Name | Formula (MW) | CAS No. | Amt. | Specification | Supplier | Description |
| sodium | [Na]₂[Au(SC₄H₃O₄)]•xH₂O | 12244-57-4 | 3.108 g | 99.9% | Alfa Aesar | white |
| aurothiomalate | (390.08 anhy.) | | | (metals | Cat No. | powder |
| hydrate | | | | basis) | 39740 | |
| Puralox 5/90 | Al₂O₃ | 1344-28-1 | 100 g | As | Sasol | white |
| Alumina | (101.96) | | | supplied | | powder |
| Distilled | H₂O | 7732-18-5 | 86 g | deionized | — | — |
| Water | (18.02) | | | | | |

### C.2—Catalyst Preparation—Target Loading of 1.5 wt % Au

1. Weigh out 100 g of Puralox 5/90 alumina.
2. Dissolve 3.1 g of sodium aurothiomalate in 86 g of distilled deionized water to form a clear, colorless solution.
3. Impregnate the Puralox 5/90 alumina by incipient wetness using the solution prepared in #2.
4. Place the impregnated material into a vacuum oven and dry at 80° C. and for one hour.
5. Calcine dried catalyst material using a ramp rate of 5°/min to 300° C. and hold at 300° C. for 4 h.

### E.—Tetrachloroauric Acid Deposition Precipitation Catalyst Prep—Powder Only

### E.1—Raw Materials

| | | | | | Purity | | |
|---|---|---|---|---|---|---|---|
| Material Name | Formula (MW) | CAS No. | Amt. | | Spec. | Supplier | Description |
| hydrogen | HAuCl₄•xH₂O | 27988-77-8 | 4.64 | | 99.9% | Strem | yellow waxy |
| tetrachloroaurate | (339.79 anhy) | | | | (metals | Chemical | crystals |
| hydrate | | | | | basis) | | |
| sodium thiosulfate | Na₂(S₂O₃)•5H₂O | 10102-17-7 | 7.54 | | 99+% | — | white |
| pentahydrate | (248.17) | | | | | | crystals |
| sodium carbonate | Na₂CO₃ | 497-19-8 | 2M aq. | | Anhydrous, | — | white |
| | (105.99 anhy) | | soln. | | 99+% | | crystals |
| Puralox 5/90 | Al₂O₃ | 1344-28-1 | 100 | g | As | Sasol | white |
| | (101.96) | | | | supplied | | powder |
| Distilled Water | H₂O | 7732-18-5 | 3.6 | L | deionized | | — |
| | (18.02) | | | | | | |

### E.2—Catalyst Preparation—Target Loading of 1.5 wt % Au

1. Add 600 mL of DI water to a suitable container equipped with a Teflon stir bar.
2. Suspend 100 g of Puralox 5/90 in the water and stir at 450 RPM.
3. Add 7.54 g of sodium thiosulfate pentahydrate to the mixture and stir for 1 h at 60° C. Note initial pH.
4. Adjust pH of the mixture by adding dropwise 2M aqueous sodium carbonate until the pH of the mixture reaches 9.95.
5. Add 4.64 g hydrogen tetrachloroaurate to the mixture and stir for an additional 1.5 h at 60° C. Note the final pH of the solution.
6. Discontinue heating and stirring and allow material to settle to the bottom of the container. Decant liquid and resuspend the solid in ˜1 L of DI water.
7. Repeat step 6 two additional times and recover the solid via filtration. Air dry overnight at ambient temperature (i.e. spread on a watch glass or other suitable container.)
8. Calcine the resulting light brown solid in a furnace using a 5° C./min ramp to 400° C., holding at 400° C. for 4 h.
9. Collect the resulting purple solid and store in an amber glass container. Keep material cold until ready for use or shipment.
Performance data for deposition precipitation prepared catalysts with sodium thiosulfate additive on high-surface-area γ-alumina:

| | | | | | | MMA | | MIB/ | Average |
|---|---|---|---|---|---|---|---|---|---|
| | | Gold | MMA | Acetal | MAA | per | MMA sel | MMA | head O₂ |
| Gold loadings | | uptake | rates | rates | rates | mole | (acetal | (ppm, | partial |
| (wt %) | | effic. | (mol/h/ | (mol/h/ | (mol/h/ | of Au | excluded, | rate | pressure |
| nominal | meas. | (%) | kg cat) | kg cat) | kg cat) | (1/s) | normalized) | ratio) | (psig) |
| 1.66 | 0.61 | 36.7 | 35.3 | 2.5 | 0.6 | 0.32 | 98.5 | 1032.0 | 1.7 |
| | | (final | | | | | | | |
| | | pH = 9.3) | | | | | | | |
| 1.33 | 0.91 | 68.4 | 37.8 | 1.8 | 0.6 | 0.23 | 98.4 | 876 | 1.9 |
| | | (final | | | | | | | |
| | | pH = 7.8) | | | | | | | |
| 1.5 | 0.72 | 47.9 | 34.8 | 1.2 | 0.5 | 0.26 | 98.6 | 645 | 1.6 |
| | | (final | | | | | | | |
| | | pH = 8.9) | | | | | | | |

### Comparative Example: Au—Ni Catalysts

| | | | | | | | | | | Avg. |
|---|---|---|---|---|---|---|---|---|---|---|
| | | | | | MMA | | | | MIB/ | head |
| nominal | | measured | | gold | rate | Acetal | MMA | MMA selec | MMA | space O₂ |
| loading | | loading | | uptake | (mol | Rate | rate/ | (%) (excl. | rate | partial |
| (wt %) | | (wt %) | | effic., | MMA/ | (mol MMA/ | mol Au | acetal, | ratio | pressure |
| Au | Ni | Au | Ni | % | kg-cat-hr) | kg-cat-hr) | (s⁻¹) | normalized) | (ppmw) | (psig) |
| 1.5 | 0.5 | 0.71 | 0.26 | 47 | 10.8 | 5.1 | 0.08 | 97.1 | 0 | 5.5 |
| 1.5 | 1.5 | 0.77 | 1.3 | 51 | 24.2 | 3.0 | 0.17 | 98.0 | 508.1 | 3.6 |
| 1.5 | 3 | 0.97 | 2.57 | 65 | 28.8 | 3.0 | 0.16 | 98.5 | 2750.6 | 1.9 |

R×n conditions: 10 wt % MA/MeOH, 8.5% O2, 100 sccm, 100 psig, 1 g catalyst, 80° C.

### Comparative Example—Catalyst 425

| | | | | | | MMA | | MIB/ | Average |
|---|---|---|---|---|---|---|---|---|---|
| | | Gold | MMA | Acetal | MAA | rates | MMA sel | MMA | head O₂ |
| Gold loadings | | uptake | rates | rates | rates | per mole | (acetal | (ppm, | partial |
| (wt %) | | effic. | (mol/h/ | (mol/h/ | (mol/h/ | of Au | excluded, | rate | pressure |
| nominal | measured | (%) | kg cat) | kg cat) | kg cat) | (1/s) | normalized) | ratio) | (psig) |
| 3.6 | 1.25 | 35 | 35.3 | 0.5 | 0.6 | 0.15 | 98.2 | 757.0 | 2.8 |
| 3.6 | 1.25 | 35 | 30.2 | 1.6 | 0.5 | 0.13 | 98.2 | 517.0 | 2.1 |

### Operation Details

### A. Typical Operation for Continuous Fixed Bed Reactor

A representative example is provided here and corresponds to the testing conditions employed for the catalyst of Example 5. The reactor consisted of 2′ (61 cm)×0.25″ (6.4 mm) stainless steel tube which was loaded with 0.38 g of catalyst dispersed in 19 g of 200 um silicon carbide fines. The reactor was heated via a jacket fed by a recirculating heater to maintain temperature. For this experiment, the most typical reaction temperature was 60° C. Synthetic air and helium were continuously fed to the reactor via separate mass flow controllers allowing the oxygen content of the gas feed to be adjusted (typically 6% O₂ in inerts). Liquid was fed concurrently via a pump and delivered a solution consisting of 10 wt % methacrolein in methanol. The reactor was operated in trickle-flow mode with both liquid and gas being fed and flowing down through the reactor during operation. The reactor was typically operated at a pressure of 160 psig (1200 kPa) which was maintained with a backpressure regulator. The effluent from the reactor then passed through a flash column consisting of a ½″ (12.7 mm) diameter stainless steel tube packed with 3 mm glass beads and maintained at a temperature of 110° C. and a pressure of 10 psig (170 kPa). An online gas chromatograph facilitated analysis of the reactor effluent stream from the flash.

### B. Typical Operation for ‘Batch Recycle’ Fixed Bed Reactor

In a typical experiment, a solution was prepared comprising 20 wt % methacrolein, 200 ppm inhibitor (4-HT), and a balance of methanol. Then, the solution was buffered by adding 0.3 wt % methacrylic acid and subsequent titration to pH 7 using 10 wt % NaOH in water. 150 g of the liquid feed was pumped into a 300 ml reactor, which served as a gas disengagement vessel. The vessel was cooled with external cooling coils maintaining a temperature ˜15-20° C. within the vessel. The liquid feed was pumped at 7 mL/min from the gas-disengagement vessel into the bottom of the vertically-oriented fixed bed reactor. Air/N₂ gas feed was mixed with the liquid feed prior to entering the fixed bed reactor. The fixed bed reactor was a ¼″ stainless steel tube (approximately 36 inches long) within a ½″ (12.7 mm) tube jacket. The inner diameter of the reactor was 0.18 inch (4.6 mm). Water that was maintained at 60° C. using an external heater was circulated through the jacket of the reactor to maintain isothermal operation. The reactor itself was packed with 2 mm glass beads to fill half of the tube length (approximately 18 inches (46 cm)), then 2 g of catalyst. The remaining void at the top of the reactor was filled with 3 mm glass beads. Liquid and gas exiting the top of the reactor were sent to a condenser. The non-condensable gases were vented, while the liquid was recycled back into the gas-disengagement vessel. Results are described in the below table. MIB is reported in ppm on a 100% MMA product basis.

### C. Typical Operation for ‘Semi-Batch’ Slurry Reactor

The semi-batch reactor system consists of a 300 mL Parr reactor which was operated as a stirred tank reactor. The gas feed is continuous while the liquid in the reactor was charged to the reactor at the beginning of an experimental run. In a typical experiment, an appropriate amount of catalyst (0.5-2 g) was charged to the reactor after which a reactant solution (typically 150 g of a 10 wt % methacrolein in methanol) was metered into the reactor by pump. Once the reactor is fully loaded, the reactor is pressurized to 100 psig (790 kPa) and this pressure was maintained. Gas was continuously introduced into the reactor by calibrated mass flow controllers capable of delivering nitrogen and air and typically feeding 8% oxygen in nitrogen. The gas was dispersed throughout the reaction mixture by means of a gas dispersing impeller rotating at 1150 RPM. The gaseous effluent was passed through a condenser to prevent the majority of the condensable components from leaving the reactor. Some organics and non-condensable gases exited the condenser and were analyzed online by a gas chromatograph. An external sample loop was used to periodically collect liquid samples from the reactor which were then analyzed to monitor the reaction progress using a separate offline gas chromatograph.

### 1. Example Recipe for Egg Shell Catalyst: Sodium Aurothiomalate on Alumina (Example 5 (Catalyst 481)—Note Examples 7 and 8 are Similar, But on a Different Size Support and as Such are not Explicitly Described Here):

An impregnation solution was prepared by dissolving 0.3108 g of sodium aurothiomalate dihydrate in 10.7812 g of deionized water. Next a 10.0617 g sample of 1/16″ diameter alumina cylinders (Norpro, H.G. 08408, H.S.A. Alumina, Surface Area=226 m²/g, Pore Diameter=122 Å, Pore Volume=0.72 cc/g) was dried in an oven at 120° C. for at least one hour to provide a dry sample. The solution of sodium aurothiomalate was applied to this dried solid until the incipient wetness point of the material was reached. The resulting material was then placed in a static drying oven for one hour at 80° C. and then placed in a box furnace with an air purge. The temperature was increased to 300° C. at a ramp rate of 5° C./min and then held at this temperature for 4 hours.

### 2. Example Recipe for Non-Egg Shell Catalyst: Sodium Aurothiosulfate on Alumina (Example 6 (Catalyst 547) Note: Example 9 is Similar, But on a Different Size Support and as Such is not Explicitly Described Here.)

An aqueous solution of sodium aurothiosulfate was prepared by dissolving 0.3837 g of this material in 9.3746 g of deionized water. The resulting material was applied to 10.1179 g of H.S.A. 1/16″ (1.6 mm) cylindrical alumina pellets identical to those used in Example 1 until the incipient wetness point was reached. The resulting material was dried for at ambient pressure and a temperature 120° C. after which it was placed in a box furnace and heated to 350° C. at a ramp rate of 5° C./min and then calcined at this temperature for four hours after which the catalyst material was ready for use.

### 3. Example Recipe for Egg Shell Catalyst: Sodium Aurothiosulfate+Thiomalic Acid Alumina (Example 10 (Catalyst 797))

The incipient wetness point for Norpro H.S.A. alumina was measured using distilled water (Norpro SA6275, 3.2 mm spheres, Lot No. 2016910048, Surface Area=238 m²/g, Pore Diameter=118 Å, Pore Volume=0.73 cc/g). A solution was prepared by dissolving sodium aurothiosulfate and mercaptosuccinic acid in deionized water. This solution was stirred for 30-45 minutes and then applied to the alumina support until the incipient wetness point was reached. The quantities used are specified in Table 2. The catalyst was placed in a box furnace with an air purge set at 50 Lph and heated at 2° C./min to 80° C., held at this temperature for 2 hours, heated at 5° C./min to 400° C. and then held at this temperature for 4 hours.

TABLE 1
Reagent Quantities for Preparation Example 3

| | | | Sodium | | | |
|---|---|---|---|---|---|---|
| | | | aurothio- | Mercapto- | | |
| | | Al₂O₃ | sulfate | succinic | Oxalic | |
| Ex. | Catalyst | Quantity | dehydrate | acid | acid | Water |
| No. | No. | (g) | (g) | (g) | (g) | (g) |
| 10 | 797 | 10 | 0.385 | 0.4 | 0 | 5 |

### 4. Example Recipe for Egg Shell Catalyst: Sodium Aurothiosulfate+Thiomalic Acid on Alumina (Examples 11-13 (Catalyst 823, 826))

The incipient wetness point for Norpro H.S.A. alumina was measured using distilled water (Norpro SA6275, 3.2 mm spheres, Lot No. 2016910048, Surface Area=238 m²/g, Pore Diameter=118 Å, Pore Volume=0.73 cc/g). A sample of 50 g of this material was added to a 1 L beaker and then soaked for 10 minutes in a solution consisting of 400 g of deionized water and 40 g of concentrated ammonium hydroxide solution (Fisher Scientific, ACS, 28-30% wt %). The solution was decanted and replaced with fresh ammonium hydroxide solution of the same composition and soaked for a second time. This too was decanted and the sample was washed with 500 mL of deionized water. The pH of the supernatant was adjusted to 5.5 with mercaptosuccinic acid and this wash was decanted. The material was washed a second time with 500 mL of deionized water, the wash was decanted, and the material was permitted to dry at ambient temperature and pressure overnight. A portion of this sample was then treated under flowing nitrogen (20-40 Lph) at 400° C. for 5 hours after having been heated to this temperature at a ramp rate of 5° C./min.

An impregnation solution was prepared by dissolving appropriate amounts of sodium aurothiosulfate and mercaptosuccinic acid in appropriate amounts of deionized water. This solution was stirred for 30 minutes at ambient temperature and pressure and then applied to a sample of the alumina described above until the incipient wetness point was reached. The material was then dried and heat treated in a box furnace with an air purge set at 50 Lph by ramping at 2° C./min to 80° C., holding at 80° C. for 2 hours and then increasing the temperature at a ramp rate of 5° C./min to 400° C. and calcining at this temperature for 4 hours. The specific quantities of reagents employed are provided in Table 2.

TABLE 2
Reagent Quantities for Catalyst Preparation Example 4

| | | | Sodium | | | |
|---|---|---|---|---|---|---|
| | | | aurothio- | Mercapto- | | |
| | | Al₂O₃ | sulfate | succinic | Oxalic | |
| Ex. | Catalyst | Quantity | dehydrate | acid | acid | Water |
| No. | No. | (g) | (g) | (g) | (g) | (g) |
| 11 | 823 | 5 | 0.19 | 0.034 | 0 | 5 |
| 12&13 | 826 | 10 | 0.385 | 0.4 | 0 | 11 |

### 5. Example Recipe for Egg Shell Catalyst: Sodium Aurothiosulfate+Other Thiol Promoter Additives (Examples 14-19, Catalysts 690, 847-877)

For Example 14 only: An aqueous solution was prepared by dissolving sodium aurothiosulfate dihydrate and mercaptosuccinic acid in deionized water. Quantities are specified in Table 3. This solution was applied to an alumina support (Norpro, H.G. 08408, H.S.A. Alumina, Surface Area=226 m²/g, Pore Diameter=122 Å, Pore Volume=0.72 cc/g) The resulting material was placed in a box furnace and calcined in flowing air (50 Lph) by ramping to 400° C. at 5° C./min and holding at this temperature for 4 h after which the material was ready for use.
For Examples 15-19: An aqueous solution was prepared by dissolving sodium aurothiosulfate dihydrate and a mercapto-containing species in deionized water and applying the resulting solution to an alumina support. (Norpro SA6275, 3.2 mm spheres, Lot No. 2016910048, Surface Area=238 m²/g, Pore Diameter=118 Å, Pore Volume=0.73 cc/g) The quantities are specified in Table 3. The material was dried at atmospheric temperature and pressure and then calcined in flowing air at 50 Lph by heating at 5° C./min to 400° C. and holding at this temperature for 4 h.

TABLE 3
Reagent Quantities for Catalyst Preparation Example 5

| | | | Sodium | | Amount of | |
|---|---|---|---|---|---|---|
| | | Al₂O₃ | aurothio- | | Mercapto | |
| | Catalyst | Quantity | sulfate | Mercapto | Species | Water |
| Ex. No. | No | (g) | dihydrate | Species Used | (g) | (g) |
| 13 | 690 | 20 | 0.82 | Mercaptosuccinic | 1.02 | 23 |
| | | | | acid | | |
| 14 | 846 | 10 | 0.38 | Thiolactic acid | 0.41 | 10 |
| 15 | 873 | 5 | 0.192 | Sodium | 0.15 | 5 |
| | | | | thioglycolate | | |
| 16 | 874 | 5 | 0.192 | 1-thioglycerol | 0.165 | 5 |
| 17 | 875 | 5 | 0.192 | Thiosalicylic acid | 0.205 | 5 |
| 18 | 877 | 5 | 0.192 | Thioglycolic acid | 0.145 | 5 |

### 6. Example Recipe for Egg Shell Catalyst: Tetrachloroauric Acid+Thiomalic Acid on Alumina (Example 20 (Catalyst 829))

A solution was prepared by dissolving 1.15 g of tetrachloroauric acid and 2.9 of mercaptosuccinic acid in 36 g of water. The solution was stirred for 60 minutes at room temperature and pressure and then applied to 35 g of 3.2 mm Norpro H.S.A. alumina spheres as described in examples 1-4. The material was placed in a fume hood and permitted to dry under ambient conditions after which it was placed inside a box oven with an air purge and dried for 10 h at 80° C.

A 10 g portion of the material prepared above was calcined by heating at 5° C./min to 300° C. and holding at this temperature for 2.5 hours using a box furnace. The resulting material was then soaked in 150 mL of an aqueous solution of 5 wt % sodium hydroxide for fifteen minutes. After this, the hydroxide solution was decanted and replaced with 150 mL of deionized water and soaked for fifteen minutes. These two steps were repeated in sequence four additional times after which the material was dried under air in a box furnace for 2 h at 80° C. and calcined as second time by heating at 5° C./min to 300° C. and holding at this temperature for 2.5 h after which the material was ready for use.

| | | | | | | Space Time | | | |
|---|---|---|---|---|---|---|---|---|---|
| | | | | MA | MMA | Yield (mol | MIB/ | Reactor | |
| Ex. | Prep. | Egg | Au | Conv. | Selectivity | MMA/ | MMA | Type | |
| No. | Method | Shell | (wt %) | (%)^{a} | (%)^{a} | kg-cat-hr) | ratio^{a} | Used | Notes |
| 5 | 1 | Yes | 1.20 | 50 | 99 | 6 | 500 | A | Catalyst prepared on |
| | | | | | | | | | Norpro H.S.A. 1/16″ |
| | | | | | | | | | Al₂O₃ cylinders |
| 6 | 2 | No | 1.10 | 25 | 99 | 3 | 495 | A | Catalyst prepared on |
| | | | | | | | | | Norpro H.S.A. 1/16″ |
| | | | | | | | | | Al₂O₃ cylinders |
| 7 | 1 | Yes | 1.42 | 80 | 99 | 11 | 350 | B | Catalyst prepared on |
| | | | | | | | | | Norpro H.S.A. |
| | | | | | | | | | Al₂O₃ 1 mm spheres |
| 8 | 1 | Yes | 1.38 | 55 | 96 | 6 | 504 | A | Catalyst prepared on |
| | | | | | | | | | Norpro H.S.A. 3.2 |
| | | | | | | | | | mm Al₂O₃ spheres |
| 9 | 2 | No | 1.21 | 10 | 90 | 1 | 780 | A | Catalyst prepared on |
| | | | | | | | | | Norpro H.S.A. 3.2 |
| | | | | | | | | | mm Al₂O₃ spheres |
| 10 | 3 | Yes | 1.43 | 40 | 88 | 4.1 | 604 | B | |
| 11 | 4 | Yes | 1.07 | 40 | 87 | 7.7 | 615 | B | Catalyst prepared on |
| | | | | | | | | | Norpro H.S.A. 3.2 |
| | | | | | | | | | mm Al₂O₃ spheres |
| 12 | 4 | Yes | 1.45 | 40 | 89 | 5.2 | 597 | B | Catalyst prepared on |
| | | | | | | | | | Norpro H.S.A. 3.2 |
| | | | | | | | | | mm Al₂O₃ spheres |
| | | | | | | | | | Au predominantly in |
| | | | | | | | | | a region 10-20 μm |
| | | | | | | | | | from catalyst surface |
| 13 | 4 | Yes | 1.45 | 30.3 | 98.9 | 26.9 | 751 | C | Tie point between |
| | | | | | | | | | Fixed Bed and |
| | | | | | | | | | Slurry modes |
| 14 | 5 | Yes | 1.52 | 25 | 99.4 | 26.7 | 490 | C | |
| 15 | 5 | Yes | 0.80 | 16 | 99.2 | 17.8 | 645 | C | |
| 16 | 5 | Yes | 1.55 | 14 | 99.3 | 16.4 | 580 | C | |
| 17 | 5 | Yes | 1.43 | 10 | 99.6 | 11.2 | 608 | C | |
| 18 | 5 | Yes | 1.36 | 11 | 99.6 | 10.5 | 546 | C | |
| 19 | 5 | Yes | 0.59 | 16 | 99.2 | 17.8 | 645 | A | MA conversion is at |
| | | | | | | | | | 2 h TOS and |
| | | | | | | | | | excludes formation |
| | | | | | | | | | of methacrolein |
| | | | | | | | | | acetal. |
| 20 | 6 | Yes | 2.09 | 74 | 98 | 3.8 | 340 | A^{d} | |

^{a}MA = methacrolein, MMA = methyl methacrylate, MAA = methacrylic acid, MIB = methylisobutyrate.
(b) Note: catalysts were tested by feeding 10 wt % methacrolein in methanol at 60° C. with a co-feed of 6% O₂ in nitrogen.
(c) Tested in continuous fixed bed reactor system. Liquid samples were collected and analyzed offline, but operationally this is similar to the description given in Example A.

1. A method for preparing a heterogeneous catalyst; said method comprising steps of:
(a) combining (i) a support, (ii) a solution of a noble metal compound and (iii) a C₂-C₁₈ thiol comprising at least one hydroxyl or carboxylic acid substituent; to form a wet particle and
(b) removing water from the wet particle by drying followed by calcination to produce the catalyst.

2. The method of claim 1 in which the C₂-C₁₈ thiol comprising at least one hydroxyl or carboxylic acid substituent has from one to three substituents selected from the group consisting of carboxylic acids and hydroxyls.

3. The method of claim 2 in which the noble metal is gold.

4. The method of claim 3 in which the support is selected from the group consisting of γ-, δ-, or θ-alumina, silica, magnesia, titania, vanadia, lanthanum oxide, ceria and combinations thereof.

5. The method of claim 4 in which the support has an aspect ratio no more than 3:1.

6. The method of claim 5 in which the C₂-C₁₈ thiol comprising at least one hydroxyl or carboxylic acid substituent has from two to eight carbon atoms.

7. The method of claim 6 in which the amount of noble metal as a percentage of noble metal and the support is from 0.2 to 5 wt %.

8. The method of claim 7 in which the wet particle is dried at a temperature from 20-150° C. under vacuum to form a dried particle and the dried particle is calcined at a temperature from 250-550° C.

9. The method of claim 8 in which average diameter of the catalyst particle is from 60 microns to 10 mm.

10. The method of claim 9 in which the C₂-C₁₈ thiol comprising at least one hydroxyl or carboxylic acid substituent is selected from the group consisting of thiomalic acid, 3-mercaptopropionic acid, thioglycolic acid, 2-mercaptoethanol, 1-thioglycerol, their conjugate bases and combinations thereof.
